(19) United States
(12) Patent Application Publication
(10) Pub. No.: US 2005/0064452 A1
(43) Pub. Date: Mar. 24, 2005

(54) System and method for the detection of analytes

(76) Inventors: Matthew J. Schmid, Austin, TX (US); C. Grant Willson, Austin, TX (US)

Correspondence Address: MEYERTONS, HOOD, KIVLIN, KOWERT & GOETZEL, P.C., P.O. BOX 398, AUSTIN, TX, 78767-0398, US

(21) Appl. No.: 10/832,469

(22) Filed: Apr. 26, 2004

Related U.S. Application Data
(60) Provisional application No. 60/465,336, filed on Apr. 25, 2003.

(51) Int. Cl. C12Q 1/68; C12P 19/34; C12M 1/34

(52) U.S. Cl. 435/6; 435/91.2; 435/287.2

(57) ABSTRACT

A system and method for the detection of an analyte using multiplexing of the sensing elements is described. In one embodiment, a sensor array includes sensing elements, and probes bound to one or more sensing elements. The sensor array is formed from a supporting member to which a plurality of sensing elements may be coupled. The sensing element may have a predefined shape, size or location. A signal may be produced when a target analyte interacts with a probe. In one embodiment, the identity of the target may be determined by the detection of the signals produced and the shapes of the sensing elements. Each analyte may be given a unique code that is represented by one or more sensing elements.

### PRIORITY CLAIM

This application claims the benefit of U.S. Provisional Application No. 60/465,336 entitled “Sensor Array for the Detection of Analytes,” filed Apr. 25, 2003.

### BACKGROUND OF THE INVENTION

1. Field of Invention

The present invention relates to a method and device for the identification of analytes. More particularly, the invention relates to the development of a system and method capable of identifying a plurality of analytes with a minimal number of sensing elements by multiplexing the sensing elements of the system.

2. Description of Related Art

Since the genomic-sequencing of more than 100 organisms first began, an almost overwhelming compendium of genetic and proteomic analytes (target-molecules, targets) have been identified. Researchers are now beset with the task of characterizing these analytes, as well as identifying and characterizing their extant natural variants. Combinatorial analytical techniques, such as microarray analysis, have emerged as preferred tools for performing these characterizations. Thus, in recent years numerous commercially available DNA-based arrays and proteomic-arrays have appeared on the market. A common attribute shared by all of these devices is the convention of distributing each probe onto its own exclusive array feature or features. This methodology imposes the requirement that the arrays possess, at minimum, an equivalent number of features as they have probes. Due to size constraints placed on the dimensions of the array, this requirement has inspired array designers to investigate higher density fabrication processes for their arrays. This pursuit has engendered designs with reduced feature sizes and/or tighter spacing between features, yet each of these strategies has its limitations. Decreasing feature sizes correspondingly reduces the amount of sensing molecules located at each feature, and depending of the sensor-platform, the extent to which feature spacing can be reduced is intractably limited by the resolution of the mechanical devices used to micro-fluidically print each of the array features (e.g., a typical value is somewhere on the order of 100 microns between features).

Likewise, the development of smart sensors capable of discriminating different analytes, toxins, and bacteria has become increasingly important for clinical, environmental, health and safety, remote sensing, military, food/beverage and chemical processing applications. Many sensors capable of high sensitivity and high selectivity detection have been fashioned for single analyte and solution phase multi-analyte detection capabilities. The advantages of such array systems are their utility for the analysis of multiple analytes and their ability to be customized to respond to detect new analytes. Such on site adaptive analysis capabilities afforded by the array structures make their utilization promising for a variety of future applications.

A method of rapid sample analysis for use in a variety of areas is desirable. For example, in many biological applications the techniques now used for rapid microbiology diagnostics detect either antigens or nucleic acids. Rapid antigen testing is based on the use of antibodies to recognize either the single cell organism or the presence of infected cell material. Inherent to this approach is the need to obtain and characterize the binding of the antibody to unique structures on the organism being tested. Since the identification and isolation of the appropriate antibodies is time consuming, these techniques are typically limited to a single agent per testing module and there is no opportunity to evaluate the amount of agent present.

Most antibody methods are relatively insensitive and require the presence of 10⁵ to 10⁷ organisms. The response time of antibody-antigen reactions in diagnostic tests of this type ranges from 10 to 120 minutes, depending on the method of detection. The fastest methods are generally agglutination reactions, but these methods are less sensitive due to difficulties in visual interpretation of the reactions. Approaches with slower reaction times include antigen recognition by antibody conjugated to either an enzyme or chromophore. These test types tend to be more sensitive, especially when spectrophotometric methods are used to determine if an antigen-antibody reaction has occurred. These detection schemes do not, however, appear to allow the simultaneous detection of multiple analytes on a single detector platform.

The alternative to antigen detection is the detection of nucleic acids. An approach for diagnostic testing with nucleic acids uses hybridization to target unique regions of the target organism. Many known techniques require fewer organisms (10³ to 10⁵), but require about five hours to complete.

The most recent improvement in the detection of microorganisms has been the use of nucleic acid amplification. Nucleic acid amplification tests have been developed that generate both qualitative and quantitative data. However, the current limitations of these testing methods are related to delays caused by specimen preparation, amplification, and detection. Currently, the standard assays require about five hours to complete. The ability to complete much faster detection for a variety of microorganisms would be of tremendous importance to military intelligence, national safety, medical, environmental, and food areas.

### SUMMARY

To meet the demand of increasing array densities without changing array dimensions, a novel platform-independent probe loading methodology has been developed. This methodology increases the array's density not by adding features to the array; rather, it increases the array's density by increasing the number of probes that may be distributed within the array's original number (quantity) of features. The technique, which may be used in a number of array applications, has been given the name (appellation) “feature-multiplexing.” As the name implies, feature-multiplexing breaks from the status quo of placing each probe on its own exclusive sensor feature(s) in order to index the array. Rather, feature-multiplexing indexes the array by placing each probe at a unique combination of array-features. Using this methodology, a given number of (n) of features may be used to deploy a much greater quantity (up to 2ⁿ-2) of probes. Because there is this exponential relationship between the number array features and the number of probes the array may accommodate, this methodology can dramatically augment the number of analytes an array may be used to detect.

Therefore, this invention has the potential of decreasing the number of experiments a researcher would need to perform in order to screen for the presence of a large set (greater than the current capacity of existing arrays) of analytes. Fewer experiments equates to preparing fewer samples and using fewer detection devices, and both of these outcomes are favorable to the industry.

In one embodiment, a system and method for analyzing analytes is described. The analytes may be DNA, RNA, proteins, enzymes, oligopeptides, antigens, antibodies, or organic molecules. The system may generate patterns that may identify the analyte. In one embodiment, the generated pattern may be associated with a unique code to facilitate the identification of the analyte.

The system, in some embodiments, is composed of a plurality of different sensing elements coupled to a supporting member. One or more probes may be coupled to each sensing element. A “probe” as used herein is any molecule that is capable of interacting with an analyte. In some embodiments, a probe may be DNA (single or double stranded), RNA, proteins, enzymes, oligopeptides, antigens, organic molecules, and/or antibodies. One or more of the probes may be selected to undergo a binding interaction with one or more analytes. A probe may bind with an analyte and undergo a spectroscopic change when bound to the analyte.

Each of the different sensing elements may be discriminated from each other using different techniques. In one embodiment, each individual sensing element may have a shape that is different from the shape of the other sensing elements. In another embodiment, each individual sensing element may have a size that is different from the size of the other sensing elements. In another embodiment, each individual sensing element may have a location on a support member of a sensor array that is different from the location of the other sensing elements. In another embodiment, sensing elements may be demarked (or indexed) by labeling each sensing element with a unique combination of dyes such that each possess a unique spectroscopic signature. Combinations of shape, size, indexing and locations may also be used to differentiate the different sensing elements. Each individual sensing element may be associated with one or more probes. Thus, the presence of a particular analyte may be determined by the observance of one or more signals from a sensing element.

In some embodiments, one or more probes may be loaded onto one or more sensing elements. Each individual sensing element may have a unique loading of one or more probes. The combination of the individual sensing elements may represent an encoding sequence of one or more analytes. The combination of sensing elements may produce a signal that may be interpreted as a code. The code, represented by the sensing elements, may be used to identify the particular analyte or analytes that are present.

In some embodiments, multiplexing may be enhanced by use of a parity-sensing element. A parity-sensing element may help identify the specific analyte that is interacting with one or more sensing elements when multiple analytes may have a similar code.

In some embodiments, the analyte may be coupled to an indicator. The indicator coupled to the analyte may produce a detectable spectroscopic signal (e.g., a fluorescent signal) when the analyte interacts with the sensing element. In some embodiments, the indicator may produce a spectroscopic signal when the target molecule binds to a probe. The spectroscopic signal may be used to identify the analyte.

### BRIEF DESCRIPTION OF THE DRAWINGS

Advantages of the present invention will become apparent to those skilled in the art with the benefit of the following detailed description of embodiment and upon reference to the accompanying drawings, in which:

FIG. 1 depicts loading-patterns for encoding sensing elements using a binary sequence;

FIG. 2 depicts an encoding scheme for all possible mutations of a reference DNA sequence;

FIG. 3 depicts an embodiment of an example of output of a multiplexed detection device; and

FIG. 4 depicts an embodiment of decoding the output of a multiplexed detection device; and

FIG. 5 depicts a hypothetical example of DNA disassociation curves.

While the invention is susceptible to various modifications and alternative forms, specific embodiments thereof are shown by way of example in the drawings and will herein be described in detail. It should be understood that the drawing and detailed description thereto are not intended to limit the invention to the particular form disclosed, but on the contrary, the intention is to cover all modifications, equivalents and alternatives falling within the spirit and scope of the present invention as defined by the appended claims.

### DETAILED DESCRIPTION OF EMBODIMENTS

Herein is described a method of multiplexing the features of an analyte detection device. Also described herein are systems that embody this method. In one embodiment, multiplexing is achieved by placing probes for multiple analytes on a single sensing element. In this manner, the analytes identity does not necessarily correspond to a single sensing element, but rather may correspond to multiple sensing elements. The identity of a specific analyte is therefore encoded by a unique pattern of the sensing elements. As used herein a “pattern” of sensing elements refers to a location, size, shape, spectroscopic signature or combinations thereof of one or more sensing elements. In this manner, the described sensor array may be used to detect a number of analytes that is greater than the number of sensing elements.

In one embodiment, a system and method for analyzing analytes is described. The analytes may be DNA, RNA, proteins, enzymes, oligopeptides, antigens, antibodies, or organic molecules. The system may generate patterns that may identify the analyte. In one embodiment, the generated pattern may be associated with a unique code to facilitate the identification of the analyte.

The system, in some embodiments, is composed of a plurality of different sensing elements coupled to a supporting member. One or more probes may be coupled to each sensing element. A “probe” as used herein is any molecule that is capable of interacting with an analyte. In some embodiments, a probe may be DNA (single or double stranded), RNA, proteins, enzymes, oligopeptides, antigens, organic molecules, and/or antibodies. One or more of the probes may be selected to undergo a binding interaction with one or more analytes. A probe may bind with an analyte and undergo a spectroscopic change when bound to the analyte.

Each of the different sensing elements may be discriminated from each other using different techniques. In one embodiment, each individual sensing element may have a shape that is different from the shape of the other sensing elements. In another embodiment, each individual sensing element may have a size that is different from the size of the other sensing elements. In another embodiment, each individual sensing element may have a location on a support member of a sensor array that is different from the location of the other sensing elements. In another embodiment, sensing elements may be demarked (or indexed) by labeling each sensing element with a unique combination of dyes such that each possess a unique spectroscopic signature. Combinations of shape, spectroscopic signature, size and locations may also be used to differentiate the different sensing elements. Each individual sensing element may be associated with one or more probes. Thus, the presence of a particular analyte may be determined by the observance of one or more signals from a sensing element.

The system may include sensing elements as described in U.S. Patent Application No. 2003-0003436 A1, “The Use of Mesoscale Self-Assembly and Recognition to Effect Delivery of Sensing Reagent for Arrayed Sensors,” to Willson et al. published on Jan. 2, 2003, which is incorporated herein by reference. The device, in some embodiments, is made of a plurality of different sensing elements coupled to a supporting member. Each of the different sensing elements may have a shape and/or size that differs from the shape and/or size of the other sensing elements. The shape and/or size of the sensing element may be associated with one or more specific probes. Thus, the presence of a particular target may be determined by the observance of a signal from a pattern of sensing element having predetermined shapes and/or sizes. This offers an advantage over conventional systems, where the location of the particle, rather than the shape and/or size of the particle, determines the identity of the target.

In some embodiments, the sensing elements in an array may be arranged in a pattern of dots. Specific patterns of dots may be associated with a specific receptor. In an embodiment, the sensing elements may be arranged in a pattern of lines. The lines may form a bar code. A particular bar code may be associated with a specific analyte.

In some embodiments, the array is a microfluidic array. The sensing element of the microfluidic array may be a particle. In some embodiments, the sensing element may be a particle positioned in a cavity in the microfluidic array. The microfluidic array may include a plurality of cavities. A probe may be coupled to a particle disposed in a microfluidic array. More than one probe may be loaded onto each particle of the microfluidic array. In this embodiment, encoding may be performed based on the location of the particles. Patterns of sensing elements based on the location of the sensing elements may be used to encode each of a plurality of analytes. Further details regarding such systems may be found in U.S. Pat. No. 6,649,403 entitled “Method of Preparing a Sensor Array” to McDevitt et al., which is incorporated herein by reference.

In certain embodiments, the device may include a support member where the sensing elements are positions on the support member where a probe is positioned. An inkjet delivery system may deposit nanoliter and/or picoliter volumes onto specific positions on a support member. In an embodiment, the specific positions on the array may be charged surfaces that inhibit movement of the deposited volumes.

In some embodiments, one or more probes may be loaded onto one or more sensing elements. Each individual sensing element may have a unique loading of one or more probes. The combination of the individual sensing elements may represent an encoding sequence of one or more analytes. The combination of sensing elements may produce a signal that may be interpreted as a code. The code, represented by the sensing elements, may be used to identify the particular analyte or analytes that are present.

Alternatively, probes may be coupled directly to a supporting member. The probes may be coupled to the supporting member in groups in predetermined locations. Each group that includes one or more probes may be considered a sensing element. Examples of coupling probes directly to a supporting member in discrete locations can be found, for example, in the following U.S. patent applications, each of which is incorporated herein by reference: U.S. Pat. Nos. 5,445,934; 5,700,637; 5,744,305; 5,945,334; 6,261,776; 6,291,183; 6,346,413; 6,399,365; and 6,610,482.

Sensing elements may be a polymer with one or more probes coupled to the polymer. A naturally occurring or synthetic probe may be bound to a polymer in order to create the particle. The polymer may be include, but is not limited to, agarous, dextrose, acrylamide, control pore glass beads, polystyrene-polyethylene glycol resin, polystyrene-divinyl benzene resin, formylpolystyrene resin, trityl-polystyrene resin, acetyl polystyrene resin, chloroacetyl polystyrene resin, aminomethyl polystyrene-divinylbenzene resin, carboxypolystyrene resin, chloromethylated polystyrene-divinylbenzene resin, hydroxymethyl polystyrene-divinylbenzene resin, 2-chlorotrityl chloride polystyrene resin, 4-benzyloxy-2′4′-dimethoxybenzhydrol resin (Rink Acid resin), triphenyl methanol polystyrene resin, diphenylmethanol resin, benzhydrol resin, succinimidyl carbonate resin, p-nitrophenyl carbonate resin, imidazole carbonate resin, polyacrylamide resin, 4-sulfamylbenzoyl-4′-methylbenzhydrylamine-resin (Safety-catch resin), 2-amino-2-(2′-nitrophenyl) propionic acid-aminomethyl resin (ANP Resin), p-benzyloxybenzyl alcohol-divinylbenzene resin (Wang resin), p-methylbenzhydrylamine-divinylbenzene resin (MBHA resin), Fmoc-2,4-dimethoxy-4′-(carboxymethyloxy)-benzhydrylamine linked to resin (Knorr resin), 4-(2′,4′-Dimethoxyphenyl-Fmoc-aminomethyl)-phenoxy resin (Rink resin), 4-hydroxymethyl-benzoyl-4′-methylbenzhydrylamine resin (HMBA-MBHA Resin), p-nitrobenzophenone oxime resin (Kaiser oxime resin), and amino-2,4-dimethoxy-4′-(carboxymethyloxy)-benzhydrylamine handle linked to 2-chlorotrityl resin (Knorr-2-chlorotrityl resin). In one embodiment, the polymer serving as a support for the probe is compatible with the solvent in which the analyte is dissolved. For example, polystyrene-divinyl benzene resin will swell within non-polar solvents, but does not significantly swell within polar solvents. Thus, polystyrene-divinyl benzene resin may be used for the analysis of analytes within non-polar solvents. Alternatively, polystyrene-polyethylene glycol resin will swell with polar solvents such as water. Polystyrene-polyethylene glycol resin may be useful for the analysis of aqueous fluids.

A variety of natural and synthetic probes may be used. The synthetic probes may come from a variety of classes including, but not limited to, polynucleotides (e.g., aptamers), peptides (e.g., enzymes and antibodies), synthetic receptors, polymeric unnatural biopolymers (e.g., polythioureas, polyguanidiniums), and imprinted polymers. Natural based synthetic probes include probes that are structurally similar to naturally occurring molecules. Some examples of natural probes include, but are not limited to, DNA (both single and double stranded), RNA, proteins, enzymes, oligopeptides, antigens, or antibodies. Polynucleotides are relatively small fragments of DNA, which may be derived by sequentially building the DNA sequence. Peptides may be synthesized from amino acids. Unnatural biopolymers have a chemical structure that is based on a natural biopolymer, but which is built from unnatural linking units.

Either natural or synthetic probes may be chosen for their ability to bind to target molecules in a specific manner. The forces that drive association/recognition between molecules include the hydrophobic effect, anion-cation attraction, and hydrogen bonding. The relative strengths of these forces depend upon factors such as the solvent dielectric properties, the shape of the host molecule, and how it complements the guest. Upon host-guest association, attractive interactions occur and the molecules stick together. The most widely used analogy for this chemical interaction is that of a “lock and key.” The fit of the key molecule (the guest) into the lock (the host) is a molecular recognition event.

A naturally occurring or synthetic probe may be bound to a polymer. In one embodiment, the polymer may have a predetermined shape in order to create the sensing element. In another embodiment, all of the sensing elements may have the same shape, but different sizes. In another embodiment, all of the sensing elements may have the same shape and size, but may be spatially located on a support member in specific regions of the support member. In one embodiment, the material used to form the polymeric resin is compatible with the solvent in which the target is dissolved. For example, PEG hydrogel resins will swell within polar solvents, but do not significantly swell within non-polar solvents. Thus, PEG-hydrogel resins may be used for the analysis of analytes within polar solvents. Techniques for the building of DNA fragments and polypeptide fragments on a polymer particle are well known. Techniques for the immobilization of naturally occurring antibodies and enzymes on a polymeric resin are also well known.

In an embodiment, the analyte molecules in the fluid may be pretreated with an indicator. Pretreatment may involve covalent attachment of an indicator to an analyte. After the indicator has been attached to the analyte, the fluid may be passed over the sensing elements. Interaction of the probes on the sensing elements with the analytes may remove the analytes from the solution. Since the analytes include an indicator, the spectroscopic properties of the indicator may be passed onto the sensing element. By analyzing the physical properties of the sensing elements after passage of an analyte stream, the presence and concentration of an analyte may be determined. The spectroscopic properties of the indicator may be fluorescent, chemiluminescent, and/or colorimetric. In one example, the analytes within a fluid may be derivatized with a fluorescent indicator before introducing the fluid to the sensing elements. As analyte molecules captured by the probes coupled to the sensing elements, the fluorescence of the sensing elements may increase. The presence of a fluorescent signal may be used to determine the presence of a specific analyte. Additionally, the strength of the fluorescence may be used to determine the amount of analyte within the stream.

In one embodiment, a detectable signal may be caused by the altering of the physical properties of an indicator ligand bound to the probe or a polymer to which the probe is attached. In one embodiment, two different indicators are attached to a probe or the polymer support. When an analyte is captured by the probe, the physical distance between the two indicators may be altered such that a change in the spectroscopic properties of the indicators is produced. A variety of fluorescent and phosphorescent indicators may be used for this sensing scheme. This process, known as Forster energy transfer, is extremely sensitive to small changes in the distance between the indicator molecules.

For example, a first fluorescent indicator (e.g., a fluorescein derivative) and a second fluorescent indictor (e.g., a rhodamine derivative) may be attached to a probe. When no analyte is present, short wavelength excitation may excite the first fluorescent indicator, which fluoresces. The short wavelength excitation, however, may cause little or no fluorescence of the second fluorescent indicator. After binding of analyte to the probe, a structural change in the probe molecule may bring the first and second fluorescent indicators closer to each other. This change in intermolecular distance may allow the excited first indicator to transfer a portion of its fluorescent energy to the second fluorescent indicator. This transfer in energy may be measured by either a drop in energy of the fluorescence of the first indicator molecule, or the detection of increased fluorescence by the second indicator molecule.

Alternatively, the first and second fluorescent indicators may initially be positioned such that short wavelength excitation, may cause fluorescence of both the first and second fluorescent indicators, as described above. After binding of an analyte to the probe, a structural change in the receptor molecule may cause the first and second fluorescent indicators to move further apart. This change in intermolecular distance may inhibit the transfer of fluorescent energy from the first indicator to the second fluorescent indicator. This change in the transfer of energy may be measured by either a drop in energy of the fluorescence of the second indicator molecule, or the detection of increased fluorescence by the first indicator molecule.

In another embodiment, an indicator may be preloaded onto the probe. An analyte may then displace the indicator to produce a change in the spectroscopic properties of the sensing element. In this case, the initial background absorbance is relatively large and decreases when the analyte is present. The indicator, in one embodiment, has a variety of spectroscopic properties that may be measured. These spectroscopic properties include, but are not limited to, ultraviolet absorption, visible absorption, infrared absorption, fluorescence, and magnetic resonance. In one embodiment, the indicator is a dye having either a strong fluorescence, a strong ultraviolet absorption, a strong visible absorption, or a combination of these physical properties. Examples of indicators include, but are not limited to, fluorescein, Cy3 fluorophore, Cy5 fluorophore, radioisotope, tetramethylrhodamine (TAMRA), carboxyfluorescein, ethidium bromide, 7-dimethylamino-4-methylcoumarin, 7-diethylamino-4-methylcoumarin, eosin, erythrosin, fluorescein, Oregon Green 488, pyrene, Rhodamine Red, tetramethylrhodamine, Texas Red, Methyl Violet, Crystal Violet, Ethyl Violet, Malachite green, Methyl Green, Alizarin Red S, Methyl Red, Neutral Red, o-cresolsulfonephthalein, o-cresolphthalein, phenolphthalein, Acridine Orange, B-naphthol, coumarin, and α-naphthionic acid. When the indicator is mixed with the probe, the probe and indicator may interact with each other such that one or more spectroscopic properties of the indicator are altered. The nature of this interaction may be a binding interaction, wherein the indicator and probe are attracted to each other with a sufficient force to allow the newly formed probe-indicator complex to function as a single unit. The binding of the indicator and probe to each other may take the form of a covalent bond, an ionic bond, a hydrogen bond, a van der Waals interaction, or a combination of these bonds.

The indicator may be chosen such that the binding strength of the indicator to the probe is less than the binding strength of the analyte to the probe. Thus, in the presence of an analyte, the binding of the indicator with the probe may be disrupted, releasing the indicator from the probe. When released, the physical properties of the indicator may be altered from those it exhibited when bound to the probe. The indicator may revert to its original structure and/or conformation, thus regaining its original physical properties. For example, if a fluorescent indicator is attached to a sensing element that includes a probe, the fluorescence of the sensing element may be strong before treatment with an analyte containing fluid. When the analyte interacts with the sensing element, the fluorescent indicator may be released. Release of the indicator may cause a decrease in the fluorescence of the sensing element, since the sensing element now has less indicator molecules associated with it.

As described above, a sensing element, in some embodiments, possesses both the ability to interact with the analyte of interest and to create a modulated signal. In one embodiment, the sensing element may include probe molecules that undergo a chemical change in the presence of the analyte of interest. This chemical change may cause a modulation in the signal produced by the sensing element. Chemical changes may include chemical reactions between the analyte and the probe. Probes may include biopolymers or organic molecules. Such chemical reactions may include, but are not limited to, cleavage reactions, oxidations, reductions, addition reactions, substitution reactions, elimination reactions, and radical reactions.

In one embodiment, the mode of action of the analyte on specific biopolymers may be taken advantage of to produce a sensing element. As used herein biopolymers refers to natural and unnatural: peptides, proteins, oligonucleotides, and oligosaccharides. In some instances, analytes, such as toxins and enzymes, will react with the biopolymer such that cleavage of the biopolymer occurs. In one embodiment, this cleavage of the biopolymer may be used to produce a detectable signal. A particle may include a biopolymer and an indicator coupled to the biopolymer. In the presence of the analyte the biopolymer may be cleaved such that the portion of the biopolymer which includes the indicator may be cleaved from the particle. The indicator is in this manner displaced from the sensing element. The signal of the sensing element will therefore change thus indicating the presence of a specific analyte.

In one embodiment, a sensing element may be customized for use as an immunoassay diagnostic tool. Immunoassays rely on the use of antibodies or antigens for the detection of a component of interest. In nature, antibodies are produced by immune cells in response to a foreign substance (generally known as the “antigen”). The antibodies produced by the immune cell in response to the antigen will typically bind only to the antigen that elicited the response. These antibodies may be collected and used as probes that are specific for the antigen that was introduced into the organism.

In many common diagnostic tests, antibodies are used to generate an antigen specific response. Generally, the antibodies are produced by injecting an antigen into an animal (e.g., a mouse, chicken, rabbit, or goat) and allowing the animal to have an immune response to the antigen. Once an animal has begun producing antibodies to the antigen, the antibodies may be removed from the animal's bodily fluids, typically an animal's blood (the serum or plasma) or from the animal's milk. Techniques for producing an immune response to antigens in animals are well known.

Once removed from the animal, the antibody may be coupled to a polymer support. The antibody may then acts as a probe for the antigen that was introduced into the animal. In this way, a variety of chemically specific probes may be produced and used for the formation of sensing elements. Once coupled to a polymer a number of well known techniques may be used for the determination of the presence of the antigen in a fluid sample. These techniques include radioimmunoassay (RIA) and enzyme immunoassays such as enzyme-linked immunosorbent assay (ELISA). ELISA testing protocols are particularly suited for the use of a solid polymer support. The ELISA test typically involves the adsorption of an antibody onto a solid support. The antigen is introduced and allowed to interact with the antibody. After the interaction is completed, a chromogenic signal generating process is performed which creates an optically detectable signal if the antigen is present. Signals may be generated using for example metal nanoparticles (e.g., gold nanoparticles) for detection using Raman scattering techniques. Alternatively, calorimetric, fluorescent, or chemiluminescent detection protocols may be used. Alternatively, the antigen may be bound to the solid support and a signal generated if the antibody is present.

In embodiments that involve oligonucleotide probes, double stranded DNA intercalating dyes may be used as indicators that are added to the test sample. In many cases, DNA intercalating dyes will produce a different spectroscopic signal in the presence of double stranded DNA compared to single stranded DNA. In one embodiment, oligonucleotide probes may be used to determine the presence of complementary oligonucleotide analytes. The probes may be composed of single stranded oligonucleotides that bind with the single stranded oligonucleotide analytes to form double stranded DNA. After the probes have been interacted with the analytes, an indicator solution may be added to the sensing elements. Alternatively, an indicator may be added to the test sample. The indicator may include one or more DNA intercalating dyes. Such dyes will produce a spectroscopic single in the presence of double stranded DNA that is distinct from any singles produced in the presence of single stranded DNA. Thus, such indicators may be used to detect the sensing elements that bind to the analytes to form double stranded DNA. Examples of DNA intercalators include, but are not limited to, ethidium bromide, SYBR-green, bisbenzimide intercalators (e.g., Hoechst 33258, 33342, 34580), picogreen, Acridine orange, 9-amino-6-chloro-2-methoxyacridine (ACMA), 4′,6-diamidino-2-phenylindole (DAPI), propidium iodide, 7-aminoactinomycin D (7-ADD), and LDS 751.

The system and method of detecting analytes using any of the multiplexed sensor array systems described above is further described with respect to the detection of single nucleotide polymorphism for DNA. While the following description is specific for DNA molecule, it should be understood that the following description may also be applied, in an analogous manner, to RNA, proteins, enzymes, oligopeptides, antigens, antibodies, and organic molecules.

Single nucleotide polymorphisms (“SNP”) are the most common form of genetic mutation in humans and are believed to account, to a large extent, for an individual's predisposition to disease, response to drugs, and reaction to the environment. SNP discovery arrays may be used to screen large subpopulations of people, as well as other organisms, for allelic variation (i.e., genetic diversity within gene sequences), thereby producing a set of data that is potentially useful in identifying prospective drug targets as well as developing individual tailored treatment strategies based on genetic factors.

Herein we describe a system and method for the analysis of one or more DNA analytes to determine genetic variations within a specific reference gene. In one embodiment, the systems described herein may be used to differentiate single nucleotide mismatches in a specified gene. The system may generate patterns that are diagnostic for both individual analytes and multiple analytes. The system, in some embodiments, may include probes that test for the presence of a combination of SNP's.

In one embodiment, the system and method described herein relates to identifying a genetic variation from a specific known reference gene. For example, all the known genetic variations of a reference biomolecule may be loaded on an array. When a sample containing an unknown analyte is analyzed by the array, a positive identification of a reference biomolecule or any variants of the reference biomolecule may be made. Several reference genes and their genetic variants may be analyzed on a single array. In some embodiments, only the most common genetic variants will be probes on the array.

The array may be configured to detect an SNP within a reference gene sequence. The array may include sensing elements that discriminate between analytes with single nucleotide mismatches. In one embodiment, sensing elements include probes that are sequences that are possible single base mutations to be screened by the array. The mutations may be transitions, transversions, insertions, and/or deletions. The analytes in the test sample may also include mutations of the reference gene sequence. The mutations of the analyte may be transitions, transversions, insertions, and/or deletions.

Sensing elements may be prepared by taking the reference gene sequence dividing it into one or more contiguous sections or blocks. The block length selected may be arbitrary. In some embodiments, the blocks may have a maximum length equal to one half the sum of the receptor gene length plus one. The length of a block may be large enough such that the target of the same length may have one or more mutations. In some embodiments, a probe length may be up to 100 bases. A probe length may be up to 25 bases. In some embodiments, probe lengths, in an effort to maximize the signal-to-noise characteristics of the array, may be selected such that they share a common melt-temperature and therefore have a common binding-affinity for their respective target-analytes. A probe length may be selected such that an analyte of the same length will not couple to the probe unless the analyte and probe are complementary. Approximately all of the probes in the array may have the same probe length. The array may contain all possible mutation sequences of a reference gene sequence.

In some embodiments, the DNA analyte will not bind with a complementary DNA probe when one or more base pairs are not complementary. In order to avoid cross-hybridizational noise, probes may be designed (by varying their length and sequence) such that they hybridize only to 100% complementary target sequences given a set of hybridization conditions (e.g., temperature, ionic strength, denaturant concentration, etc.). Thus, the device may include probes that represent all the potential SNP's in a reference sequence so that the target-analytes sequence can be accurately identified.

Each probe in the mutant set may be numbered and given a binary expression corresponding to the number. A binary expression may be a sequence of binary bits. A binary bit may be a “1” or a “0.” Each mutant sequence, and hence each probe, may be associated with a specific binary representation.

In a sensor array system for determining the identity of SNPs in a test sample, sensing elements may have chemically bound probes composed of single stranded DNA for complementary hybridization sensing. Oligonucleotides may be synthesized using standard methods for automated DNA synthesis with nucleoside phosphoramidites. One or more probes may be coupled to a sensing element. In some embodiments, the probes are loaded on the sensing element. Sensing elements may have a variety of shapes. The sensing elements may have unique shapes with respect to a block of the reference gene sequence. A probe may be loaded onto one or more sensing elements. Each probe may have a unique loading combination among the sensing elements.

There may be an exponential relationship between the number of sensing elements in the array (n) and the number of probe sequences the array may uniquely encode (2ⁿ). In some embodiments, the addition of additional sensing elements to the array will further enhance the degree of multiplexing. In an embodiment, binary sequences of all 1's and all 0's are not uniquely associated with a receptor. Binary sequences of all 1's or all 0's may indicate complications in the detection of a target. In an embodiment, the relationship between the number of sensing elements in the array (n) and the number of receptor sequences it can uniquely encode is 2ⁿ-2.

The ability of the sensing elements to discriminate between sequences containing single base pair mismatches in a binary manner may be used to multiplex the sensing element features. Multiplexing decreases the total number of sensing elements within a sensor array required for accurate identification of an analyte. Some DNA sensor arrays are limited spatially by the number of features that can be squeezed into a single array and limited in the number of target sequences for which they can query with a single array. Sensing element multiplexing may be used to increase the number of sequence that may be detected using a single DNA microarray.

In some embodiments, a binary sequence may not be unique to a single analyte. In some embodiments, a binary sequence may be associated with two or more analytes. A parity bit may be used to distinguish between analytes associated with identical binary sequences. The sensing elements may include one or more sensing elements that are acting as parity bits. The parity bits may increase the multiplexing capabilities of the array. Like other sensing elements, the parity bit may include one or more probes. In some embodiments, the parity bit may not include more than one probe with identical associated binary sequences. A signal associated with the parity bit may identify which probe the analyte hybridized when two or more analytes have the same binary code.

The array may be scanned or imaged to determine the array pattern. Fluorescence scanning may be used to determine the sensing elements where the analyte has hybridized with a probe. Alternatively, a microscope or CCD camera coupled to a microscope may be used to determine which sensing elements are interacting with the analyte.

In order to do high-fidelity SNP-detection-assays it is desirable to avoid cross-hybridizational noise, a phenomenon that may be defined as the unwanted hybridization between a probe and a fluorescently labeled target-sequence containing one or more base-pair mismatches. Therefore, in some embodiments, probes are designed with two requirements in mind. Probes are designed that have a high-affinity for their complement, so as to produce a large “signal” when their complement is present, while at the same time have a low-affinity for target sequences containing mismatches in order to minimize cross-hybridizational “noise”.

To be quantitative about the terms signal and noise, one must first establish a metric for the binding-affinity a probe has for its target. For DNA a well-established metric for the binding and duplex formation, called melt-temperature (Tm), already exists. The term “melt-temperature” refers to the process in which the double-helix dissociates into its two single-strands. Although the phrase melt-temperature might suggest that dissociation proceeds in a concerted manner all at a discrete temperature, this process, much like the phase transformations of non-biological polymers, progresses in a graded manner over a broad range of temperatures. Hence, the melt-temperature is defined as the temperature one must heat an aqueous solution of double-stranded DNA (dsDNA) in order to get one-half of the DNA to dissociate into its single-stranded form.

Because an oligonucleotide's melt-temperature describes the midpoint of its melting process, it serves as an excellent metric with which to gage an oligonucleotide's gross dissociation-characteristics as well as the overall affinity it has for its complement. Thus, probes which exhibit high melt-temperatures when bound to their complements, bind their complements more strongly than those that exhibit low melt-temperatures.

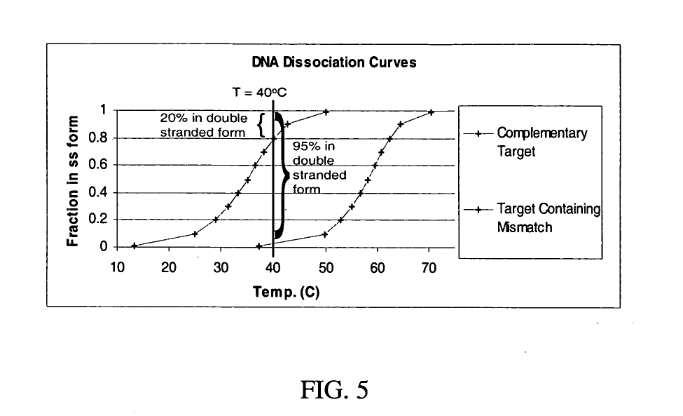

For a sensor to discriminate between two target-sequences, the hybridization-assay must be conducted at a temperature/condition that exploits the difference in the binding characteristics of the probe with respect to complementary and mismatched analytes. A hypothetical situation is depicted in FIG. 5 that shows melting point curves for a probe interacting with a complementary analyte and a mismatched analyte. In the situation depicted in FIG. 5, if a hybridization-assay is run, for example, at 40° C., the complementary target may bind strongly to the probe; in fact, the dissociation curve indicates a large fraction, over 95%, would be in the double-stranded form with the probe. However, at this temperature the sensor would also bind to a fair extent, approximately 20% in the double-stranded form, with the target containing a mismatch. Thus, for this hybridization condition, the probe, by virtue of having an abundance of cross-hybridizational noise, would have a difficult time resolving the difference between the two target analytes. Under this situation, running the detection assay at a higher temperature may improve the signal to noise ratio. In the hypothetical situation described in FIG. 5, running the analysis at 50° C. may provide a significant improvement in the signal to noise ratio. At this temperature, the probe binds strongly with the complementary target, with 90% in the double-stranded form, but not at all with the target containing a mismatch. This is because the binding strength of the probe with the mismatched analyte is insufficient to overcome the disassociation of the probe from the mismatched analyte due to the elevated temperature.

Additionally, since there are potentially thousands of probes in a single device, all being subjected to the same hybridization conditions, it is necessary that all probes be designed such that they have roughly the same dissociation-characteristics as each other. In one embodiment, this may be achieved by choosing probes that have matched melt-temperatures.

Before one can select/design the device's probes such that they have matched melt-temperatures, one must first establish a method of either measuring or predicting the melt-temperatures of prospective probe-sequences. Although there are well-established experimental techniques for spectroscopically measuring an oligonucleotide's melt-temperature, the sheer magnitude of prospective probe sequences one would need to synthesize and test make predictive models a more attractive alternative. One of the most accurate and complete models is called the “Nearest-Neighbor-Model.” This model uses empirically determined thermodynamic parameters to predict nucleic-acid duplex stability and helix propagation. One of the strengths of this model is that it may accurately predict the interaction-energies between, and hence melt-temperatures of, sequences that contain base-pair mismatches. Thus, it is possible to use this model not only to predict the signal-strength of a probe detecting its complement, but also the noise a probe would produce due to cross-hybridization. Using the Nearest-Neighbor-Model, an algorithm was developed to automate the process of selecting probes for the array such that the array's signal-to-noise characteristics were optimized.

### EXAMPLES

Materials. Poly(ethylene glycol) diacrylate (PEG-da) with a weight average molecular weight of 10,000 was purchased from SunBio. Darocur 1173, a liquid, photoinitiated free-radical generator, was obtained from Ciba-Geigy. Functionalized single stranded DNA sequences for the single-nucleotide-polymorphism (SNP) detection experiment were purchased from Integrated DNA Technologies.

Pre-polymer Formulations. Sensors for the DNA detection assay were made from a pre-polymer solution composed of (all percentages listed by volume) 25% PEG-da 10,000, 2% Darocur 1173 photoinitiator, and 73% DI water containing probe DNA. For reference-features the pre-polymer solution had a probe concentration of 30□M; for pre-polymer formulations with more than one probe type each probe's final concentration in the pre-polymer was 30 μM.

Exposure Tool & Hydrogel Fabrication. Broadband ultraviolet radiation from a 200 W high-pressure mercury arc lamp (Oriel) was used for curing. The bulb was housed in an Oriel shutter enclosure that collimated the radiation to approximately a 15 cm diameter area and filtered out wavelengths below 365 nm. The nominal intensity of the collimated light was 20 mW/cm², as measured by a Molectron PowerMax 5200 intensity meter. An Oriel 68810 Arc Lamp Power Supply, coupled with an Oriel 68705 igniter, was used to power the bulb. An Oriel 8160 Timer controlled the shutter. Hydrogel pre-polymer was irradiated for 90 seconds to fabricate sensors. Sensors were rinsed with DI water after exposure to remove any unreacted pre-polymer and stored until use in the hydrated state.

Hybridization Media for Detection-assay. The DNA detection assay was conducted in a hybridization mixture composed of 150 μl of the following constituents in DI water: 30 mM Tris (pH7.4), 450 mM NaCl, 3 mM EDTA, 7.5M formamide, and 60M of target-DNA. The sensors for the DNA assay were rinsed in a buffer solution including the same ingredients as the hybridization mixture with 3% by volume of Tween 20™ added to the solution in lieu of the target-DNA.

Imaging. An Olympus IX-71 inverted microscope was used for fluorescent imaging. A 100 W high-pressure mercury arc lamp driven by an Olympus BHL-RFL-T3 power supply served as the light source for the microscope. A 4× objective coupled with a 10×eyepiece (total 40× magnification) was used for all images. An Olympus MagnaFire SP digital camera was used to record the fluorescent micrographs. The Cy3™ fluorophore, the label on the target-DNA, was imaged with a custom filter set ordered from Leeds Instruments (catalog # 41007).

Results: To validate the methodology of feature-multiplexing, a multiplexed SNP-detection array was constructed based on shape differentiated sensing particles and tested in three SNP-detection assays. This array included eight uniquely shaped sensing elements. Five of the sensing elements were multiplexed-features, two were reference-features, and one was a control-feature.

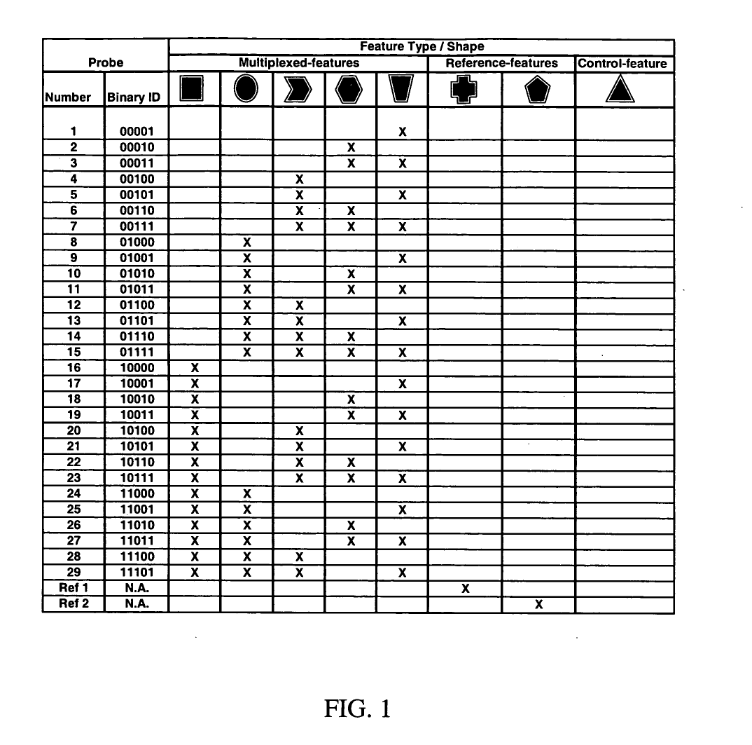

The five multiplexed-features included a total of 29 probes that were capable of screening for the presence of 29 different missense point-mutations in the human P53 gene. In order to distribute the probes properly amongst the multiplexed-features, each of the probes was given a unique feature-loading code. This was achieved by first numbering each of the probes (numbers 1-29). Next, each of the probes was assigned a code (in this example a binary-expression) corresponding to its number. Finally, the pattern of l's in the binary-expression was used to denote the unique combination of sensing elements in which the probe was to be placed. The probe loading patterns used in this demonstration are shown in FIG. 1. This figure shows the loading-patterns for each of the probes used in the array. Notice that the pattern of l's in each probe's binary ID corresponds to its unique distribution pattern amongst the multiplexed-features.

The two reference-features were not multiplexed; each contained only a single probe. The probes contained within the reference-features were each identical in sequence to a different, yet adjacent, section of the P53 reference-gene-sequence (see FIG. 1). The control-feature contained no probe at all.

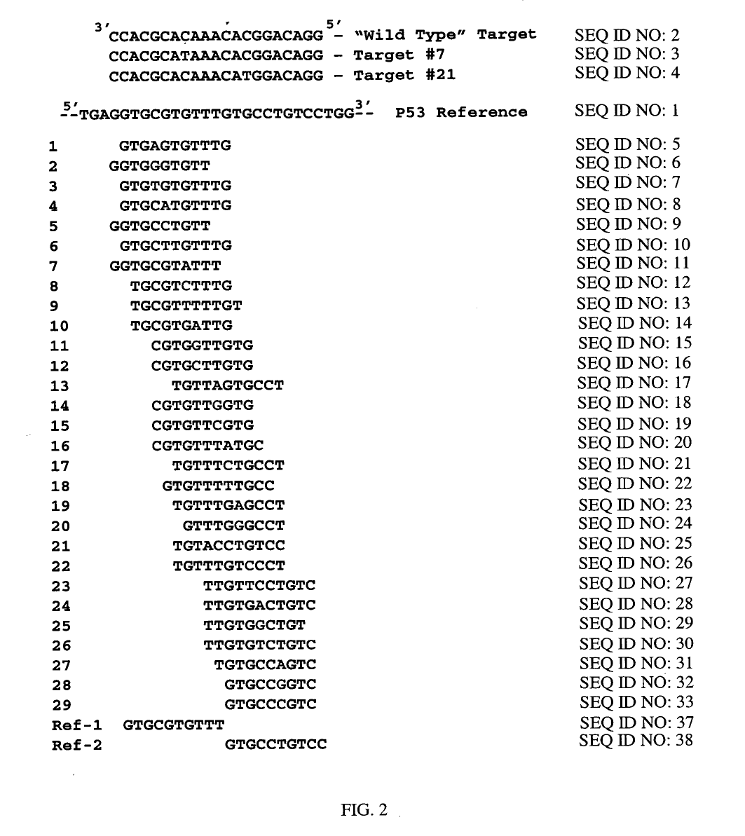

The detection-assays were conducted by placing the array-features in a buffer solution containing a fluorescently-labeled target-sequence. A different target-sequence was used for each assay. A list containing all of the target-sequences as well as probe-sequences used in the verification-of-principle study is shown in FIG. 2. This table lists all of the probe-sequences and target-sequences used in the verification-of-concept study, however not shown are the 5′-methacrylamide modifications made to the probes and the 5′-Cy3™ (a commercially available fluorophore) modifications made to each of the target-sequences. The probes and target-sequences have been aligned with respect to the P53 reference gene sequence so as to illustrate the particular region of that sequence for which they correspond. The nucleotide located in each probe that corresponds to its SNP is highlighted. Likewise, the two target-sequences that contain SNP's also have their mutant bases print highlighted.

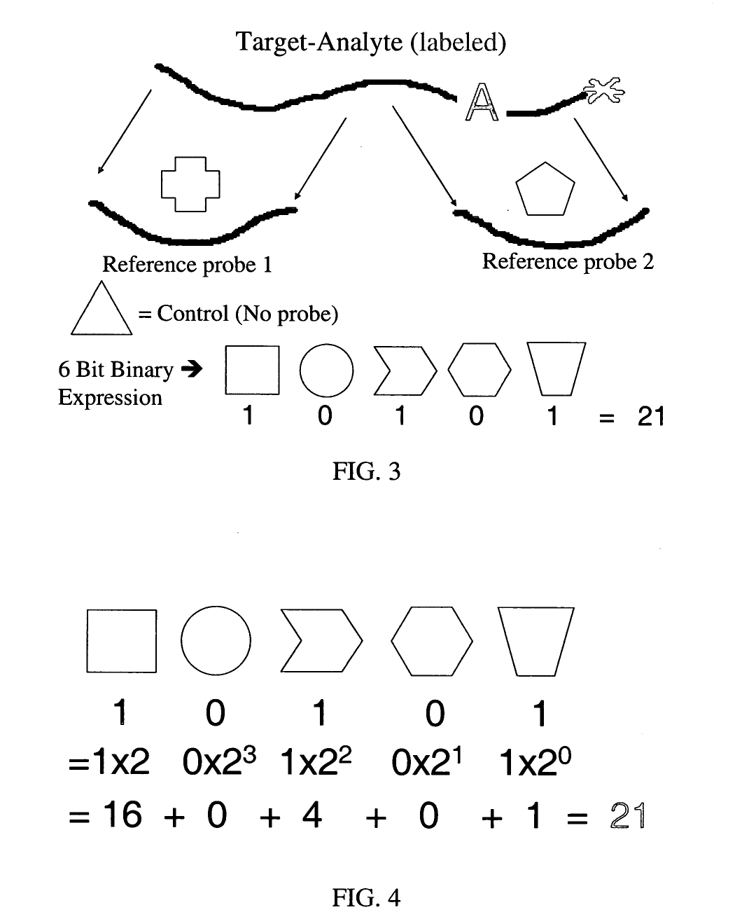

After the array-features had hybridized with the target-analytes, they were rinsed and imaged on a fluorescence-microscope. FIG. 3 illustrates how to decode the array's output received from a detection-assay. The reference-features correspond to different halves of the target-analyte (the asterisk on the right hand side of the target-sequence denotes a fluorescent-tag; the large “A” placed in the middle of the target-strand denotes a mutation that is present in the sequence). The output from the two reference-features indicates which subsection of the target contains a mutation, yet their output does not identify which mutation. The output from the multiplexed-features is what is used to identify which mutation is present. In this illustration the array is responding to a G→A mutation corresponding to probe #21.

FIG. 4 shows how the output coming from the multiplexed-features is decoded to determine the sequence of the target analyte. The pattern of fluorescing features is converted to a binary expression. The binary-expression corresponds to the number of the probe that has detected its complement. In this example, the binary code corresponds to mutation #21. Referring to FIG. 2, the mutation corresponding to code # 21 is looked up to reveal the identity of the mutation. In this case the mutation corresponds to One of the benefits of using this binary-number encryption strategy is that it simplifies the process of decoding the array's output. The pattern of fluorescing array-features is converted to a binary-expression. Each feature that fluoresces counts as a “1”; features that do not fluoresce count as a “0”. The binary-expression is then used to identify the probe that has successfully detected its complement.

In this patent, certain U.S. patents, U.S. patent applications, and other materials (e.g., articles) have been incorporated by reference. The text of such U.S. patents, U.S. patent applications, and other materials is, however, only incorporated by reference to the extent that no conflict exists between such text and the other statements and drawings set forth herein. In the event of such conflict, then any such conflicting text in such incorporated by reference U.S. patents, U.S. patent applications, and other materials is specifically not incorporated by reference in this patent.

Further modifications and alternative embodiments of various aspects of the invention may be apparent to those skilled in the art in view of this description. Accordingly, this description is to be construed as illustrative only and is for the purpose of teaching those skilled in the art the general manner of carrying out the invention. It is to be understood that the forms of the invention shown and described herein are to be taken as the presently preferred embodiments. Elements and materials may be substituted for those illustrated and described herein, parts and processes may be reversed, and certain features of the invention may be utilized independently, all as would be apparent to one skilled in the art after having the benefit of this description to the invention. Changes may be made in the elements described herein without departing from the spirit and scope of the invention as described in the following claims. In addition, it is to be understood that features described herein independently may, in certain embodiments, be combined.

1. An analyte detection device comprising a plurality of sensing elements, wherein one or more probes are coupled to each of the sensing elements, and wherein at least one of the probes is configured to interact with an analyte, and wherein at least one sensing element is configured to produce a signal when the analyte interacts with a probe coupled to the sensing element, and wherein the sensing elements produce detectable signals in a predetermined pattern, and wherein the predetermined pattern represents a code that identifies the analyte.

2. The device of claim 1, wherein the sensing elements are positioned on a supporting member at predetermined locations.

3. The device of claim 1, wherein the sensing elements are particles positioned in cavities formed on a supporting member at predetermined locations of the supporting member.

4. The device of claim 1, wherein the sensing elements are positioned on a supporting member in an array.

5. The device of claim 1, wherein each of the sensing elements comprise one or more probes coupled to a supporting member at discrete predetermined locations.

6. The device of claim 1, wherein each of the sensing elements has a shape that is different from the other sensing elements.

7. The device of claim 1, wherein each of the sensing elements has a size that is different from the other sensing elements.

8. The device of claim 1, wherein each of the sensing elements has a shape and/or size that is different from the other sensing elements.

9. The device of claim 1, wherein each of the sensing elements is capable of interacting with more than one analyte.

10. The device of claim 1, wherein each of the sensing elements comprises a polymer, wherein one or more probes are coupled to the polymer.

11. The device of claim 1, wherein each of the sensing elements comprises a mixture of one or more probes in a liquid medium.

12. The device of claim 1, wherein one or more probes comprise a nucleic acid.

13. The device of claim 1, wherein one or more probes comprise deoxyribonucleic acid.

14. The device of claim 1, wherein one or more probes comprise single stranded deoxyribonucleic acid.

15. The device of claim 1, wherein one or more probes comprise ribonucleic acid.

16. The device of claim 1, wherein one or more probes comprise an oligonucleotide.

17. The device of claim 1, wherein one or more probes comprise an oligopeptide.

18. The device of claim 1, wherein one or more probes comprise an oligonucleotide complementary with one or more single nucleotide polymorphisms of a reference DNA sequence.

19. The device of claim 1, wherein one or more probes comprise an oligonucleotide complementary with one or more single nucleotide polymorphisms of a reference DNA sequence.

20. The device of claim 1, wherein the analyte detection device is capable of determining the identify of more analytes than sensing elements.

21. The device of claim 1, wherein one or more probes comprise an oligonucleotide complementary with one or more single nucleotide polymorphisms of a reference DNA sequence, and wherein the number of sensing elements in the system is less than the number of possible single nucleotide polymorphisms of the reference DNA sequence, and wherein the analyte detection device is capable of determining the presence of analytes having all possible single nucleotide polymorphisms of a reference DNA sequence.

22. The device of claim 1, wherein the predetermined pattern represents a binary code.

23. The device of claim 1, wherein the number of analytes detectable by the analyte detection device is 2ⁿ where n is the number of sensing elements.

24. The device of claim 1, wherein the number of analytes detectable by the analyte detection device is 2ⁿ-2, where n is the number of sensing elements.

25. A method of identifying an analyte, comprising:
adding a sample that comprises one or more analytes to an analyte detection device, wherein the analyte detection device comprises:
a plurality of sensing elements, wherein one or more probes are coupled to each of the sensing elements, and wherein at least one of the probes is configured to interact with an analyte, and wherein at least one sensing element is configured to produce a signal when the analyte interacts with a probe coupled to the sensing element;
determining a pattern produced by one or more sensing elements producing a signal;
determining the code represented by the pattern; and
determining the identity of the analyte based on the determined code.

26. The method of claim 25, further comprising adding a tag to one or more analytes in the sample.

27. The method of claim 25, further comprising adding a tag to one or more analytes in the sample, wherein the tag is a fluorescent tag.

28. The method of claim 25, further comprising adding a visualization agent after the sample has been added to the analyte detection device.

29. The method of claim 25, wherein determining the code represented by the pattern comprises looking at the sensing elements with a magnification device.

30. The method of claim 25, wherein the sensing elements are positioned on a supporting member at predetermined locations.

31. The method of claim 25, wherein the sensing elements are particles positioned in cavities formed on a supporting member at predetermined locations of the supporting member.

32. The method of claim 25, wherein the sensing elements are positioned on a supporting member in an array.

33. The method of claim 25, wherein each of the sensing elements comprise one or more probes coupled to a supporting member at discrete predetermined locations, and wherein determining the code represented by the pattern comprises determining the location of the sensing elements producing a signal.

34. The method of claim 25, wherein each of the sensing elements has a shape that is different from the other sensing elements, and wherein determining a pattern comprises determining the shape of each of the sensing elements producing a signal.

35. The method of claim 25, wherein each of the sensing elements has a size that is different from the other sensing elements, and wherein determining a pattern comprises determining the size of each of the sensing elements producing a signal.

36. The method of claim 25, wherein each of the sensing elements has a shape and/or size that is different from the other sensing elements, and wherein determining a pattern comprises determining the shape and/or size of each of the sensing elements producing a signal.

37. The method of claim 25, wherein each of the sensing elements is capable of interacting with more than one analyte.

38. The method of claim 25, wherein each of the sensing elements comprises a polymer, wherein one or more probes are coupled to the polymer.

39. The method of claim 25, wherein each of the sensing elements comprises a mixture of one or more probes in a liquid medium.

40. The method of claim 25, wherein one or more probes comprise a nucleic acid.

41. The method of claim 25, wherein one or more probes comprise deoxyribonucleic acid.

42. The method of claim 25, wherein one or more probes comprise single stranded deoxyribonucleic acid.

43. The method of claim 25, wherein one or more probes comprise ribonucleic acid.

44. The method of claim 25, wherein one or more probes comprise an oligonucleotide.

45. The method of claim 25, wherein one or more probes comprise an oligopeptide.

46. The method of claim 25, wherein one or more probes comprise an oligonucleotide complementary with one or more single nucleotide polymorphisms of a reference DNA sequence.

47. The method of claim 25, wherein the analyte detection device is capable of determining the identify of more analytes than sensing elements.

48. The method of claim 25, wherein one or more probes comprise an oligonucleotide complementary with one or more single nucleotide polymorphisms of a reference DNA sequence, and wherein the number of sensing elements in the system is less than the number of possible single nucleotide polymorphisms of the reference DNA sequence, and wherein the analyte detection device is capable of determining the presence of analytes having all possible single nucleotide polymorphisms of a reference DNA sequence.

49. The method of claim 25, wherein the predetermined pattern represents a binary code.

50. The method of claim 25, wherein the number of analytes detectable by the analyte detection device is 2ⁿ, where n is the number of sensing elements.

51. The method of claim 25, wherein the number of analytes detectable by the analyte detection device is 2ⁿ-2, where n is the number of sensing elements.

52. An analyte detection device comprising a plurality of sensing elements, wherein one or more oligonucleotide probes are coupled to each of the sensing elements, and wherein at least one of the oligonucleotide probes is configured to interact with an oligonucleotide analyte, and wherein at least one sensing element is configured to produce a signal when the oligonucleotide analyte interacts with an oligonucleotide probe coupled to the sensing element, and wherein the sensing elements produce detectable signals in a predetermined pattern, and wherein the predetermined pattern represents a code that identifies the oligonucleotide analyte.

53-96. (canceled)

97. An analyte detection device comprising a plurality of groups of oligonucleotide probes coupled to a supporting member, each group of oligonucleotide probes coupled to a discrete location of the supporting member, and wherein at least one group of oligonucleotide probes are configured to produce a signal when an oligonucleotide analyte interacts with at least one of the oligonucleotide probes in the group, and wherein one or more groups of oligonucleotide probes produces a detectable signal in a predetermined pattern, and wherein the predetermined pattern represents a code that identifies the oligonucleotide analyte.

98. An analyte detection device comprising a plurality of sensing elements, each of the sensing elements having a shape that differs from the other sensing elements, wherein one or more probes are coupled to each of the sensing elements, and wherein at least one of the probes is configured to interact with an analyte, and wherein at least one sensing element is configured to produce a signal when the analyte interacts with a probe coupled to the sensing element, and wherein the sensing elements produce detectable signals in a predetermined pattern, and wherein the predetermined pattern represents a code that identifies the analyte.

99-143. (canceled)


SEQUENCE LISTING (35 sequences)

SEQ ID NO: 1 - DNA, 27 bp, Homo sapiens
tgaggtgcgtgtttgtgcctgtcctgg

SEQ ID NO: 2 - DNA, 21 bp, Homo sapiens
ccacgcacaaacacggacagg

SEQ ID NO: 3 - DNA, 21 bp, Homo sapiens
ccacgcataaacacggacagg

SEQ ID NO: 4 - DNA, 21 bp, Homo sapiens
ccacgcacaaacatggacagg

SEQ ID NO: 5 - DNA, 11 bp, Homo sapiens
gtgagtgtttg

SEQ ID NO: 6 - DNA, 10 bp, Homo sapiens
ggtgggtgtt

SEQ ID NO: 7 - DNA, 11 bp, Homo sapiens
gtgtgtgtttg

SEQ ID NO: 8 - DNA, 11 bp, Homo sapiens
gtgcatgtttg

SEQ ID NO: 9 - DNA, 10 bp, Homo sapiens
ggtgcctgtt

SEQ ID NO: 10 - DNA, 11 bp, Homo sapiens
gtgcttgtttg

SEQ ID NO: 11 - DNA, 11 bp, Homo sapiens
ggtgcgtattt

SEQ ID NO: 12 - DNA, 10 bp, Homo sapiens
tgcgtctttg

SEQ ID NO: 13 - DNA, 11 bp, Homo sapiens
tgcgtttttgt

SEQ ID NO: 14 - DNA, 10 bp, Homo sapiens
tgcgtgattg

SEQ ID NO: 15 - DNA, 10 bp, Homo sapiens
gctggttgtg

SEQ ID NO: 16 - DNA, 10 bp, Homo sapiens
cgtgcttgtg

SEQ ID NO: 17 - DNA, 11 bp, Homo sapiens
tgttagtgcct

SEQ ID NO: 18 - DNA, 10 bp, Homo sapiens
cgtgttggtg

SEQ ID NO: 19 - DNA, 10 bp, Homo sapiens
cgtgttcgtg

SEQ ID NO: 20 - DNA, 11 bp, Homo sapiens
cgtgtttatgc

SEQ ID NO: 21 - DNA, 11 bp, Homo sapiens
tgtttctgcct

SEQ ID NO: 22 - DNA, 11 bp, Homo sapiens
gtgtttttgcc

SEQ ID NO: 23 - DNA, 11 bp, Homo sapiens
tgtttgagcct

SEQ ID NO: 24 - DNA, 10 bp, Homo sapiens
gtttgggcct

SEQ ID NO: 25 - DNA, 11 bp, Homo sapiens
tgtacctgtcc

SEQ ID NO: 26 - DNA, 11 bp, Homo sapiens
tgtttgtccct

SEQ ID NO: 27 - DNA, 11 bp, Homo sapiens
ttgttcctgtc

SEQ ID NO: 28 - DNA, 11 bp, Homo sapiens
ttgtgactgtc

SEQ ID NO: 29 - DNA, 10 bp, Homo sapiens
ttgtggctgt

SEQ ID NO: 30 - DNA, 11 bp, Homo sapiens
ttgtgtctgtc

SEQ ID NO: 31 - DNA, 10 bp, Homo sapiens
tgtgccagtc

SEQ ID NO: 32 - DNA, 9 bp, Homo sapiens
gtgccggtc

SEQ ID NO: 33 - DNA, 9 bp, Homo sapiens
gtgcccgtc

SEQ ID NO: 34 - DNA, 10 bp, Homo sapiens
gtgcgtgttt

SEQ ID NO: 35 - DNA, 10 bp, Homo sapiens
gtgcctgtcc